(19) United States
(12) Patent Application Publication
(10) Pub. No.: US 2008/0095830 A1
(43) Pub. Date: Apr. 24, 2008

(54) METHOD FOR MAKING A DRESSING

(76) Inventor: Robert W. Van Holten, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/551,270

(22) Filed: Oct. 20, 2006

(51) Int. Cl. A61L 15/00; A61K 38/48

(52) U.S. Cl. 424/445; 424/94.64

(57) ABSTRACT

The present invention is directed to a method of making a dressing having at least one protein, comprising the steps of applying at least one protein to a dressing via conventional means; and further subjecting the dressing having the protein thereon to pressure ranging from about 2,500 to about 39,500 psi for about 2 to about 6 seconds.

### FIELD OF THE INVENTION

The present invention relates to a method for making a dressing.

### BACKGROUND OF THE INVENTION

The control of bleeding as well as sealing of air and various bodily fluids is essential and critical in surgical procedures to minimize blood loss, to seal tissue and organ structures, to reduce post-surgical complications, to shorten the duration of the surgery in the operating room, and to reduce mortality.

In an effort to provide dressings with enhanced hemostatic and tissue sealing and adhering properties, therapeutic agents and/or proteins, including but not limited to thrombin, fibrinogen and fibrin, have been combined with dressing carriers or substrates, including gelatin-based carriers, polysaccharide-based carriers, glycolic acid or lactic acid-based carriers, and a collagen matrix. Examples of such dressings are disclosed in U.S. Pat. No. 6,762,336, U.S. Pat. No. 6,733,774 and PCT publication WO 2004/064878 A1. Conventional means for preparing such dressings include spraying a suspension of the therapeutic agents and/or proteins onto the carrier or substrate, or dipping the carrier or substrate into a suspension of the therapeutic agents and/or proteins.

However, one major problem that persists with dressings described in the prior art having proteins thereon is the fixation of the proteins on the dressing carrier or substrate. For example, U.S. Pat. No. 7,052,713 indicates that an objective thereof is to provide a collagen sponge coated with a suspension of fibrinogen and thrombin, having a sufficient fixation of the coating to the collagen sponge. This reference further defines sufficient fixation as a satisfactory low abrasion of the coating when submitted to mechanical impact.

Additionally, it is known that pressure exerted on proteins, such as thrombin, fibrin and fibrinogen, can have a detrimental effect on the native state and the function of the proteins. “Native state” as used herein refers to the conformation of the protein that displays biological activity, which is the result of a delicate balance between stabilizing and destabilizing interactions within the protein polypeptide chains and between the protein and its environment. Pressure has been used to change the physicochemical and biochemical characteristics of a large array of proteins. For instance, some typical examples of how pressure affects the tertiary structure of proteins, i.e., induce unfolding, are discussed by Marchal et al. Braz J Med Biological Research August 2005, Vol. 38 (08) 1175-1183.

It has been found that a dressing or substrate having proteins thereon may be prepared by applying the proteins to the dressing via conventional means; and further subjecting the dressing having the proteins thereon to pressure ranging from about 2,500 to about 39,500 psi for about 2 to about 6 seconds, without affecting the physicochemical and biochemical characteristics of the proteins.

### SUMMARY OF THE INVENTION

The present invention is directed to a method for making a dressing comprising at least one protein comprising the steps of applying at least one protein to the dressing via conventional means; and further subjecting the dressing having the protein thereon to pressure ranging from about 2,500 to about 39,500 psi for about 2 to about 6 seconds.

### DETAILED DESCRIPTION OF THE INVENTION

The dressings described herein provide and maintain effective hemostasis when applied to a wound requiring hemostasis. Effective hemostasis, as used herein, is the ability to control and/or abate capillary, venous, or arterial bleeding within an effective time, as recognized by those skilled in the art of hemostasis. Further indications of effective hemostasis may be provided by governmental regulatory standards and the like.

In certain embodiments, dressings of the present invention are effective in providing and maintaining hemostasis in cases of severe or brisk bleeding. As used herein, severe bleeding is meant to include those cases of bleeding where a relatively high volume of blood is lost at a relatively high rate. Examples of severe bleeding include, without limitation, bleeding due to arterial puncture, liver resection, blunt liver trauma, blunt spleen trauma, aortic aneurysm, bleeding from patients with over-anticoagulation, or bleeding from patients with coagulopathies, such as hemophilia.

The dressings described herein may comprise absorbable or nonabsorbable polysaccharide-based carriers, absorbable or nonabsorbable polymeric-based carriers, gelatin-based carriers, or a collagen matrix. Preferably, the dressings comprise at least one knitted, woven or nonwoven fabric, a gelatin sponge or a collagen sponge.

In one embodiment, the dressing generally comprises a nonwoven fabric, wherein one or more protein, including but not limited to thrombin and/or fibrinogen, is substantially homogeneously dispersed throughout the nonwoven fabric and/or are disposed on the surface of the nonwoven fabric. As used herein, the term “nonwoven fabric” includes, but is not limited to, bonded fabrics, formed fabrics, or engineered fabrics, that are manufactured by processes other than weaving or knitting. More specifically, the term “nonwoven fabric” refers to a porous, textile-like material, usually in flat sheet form, composed primarily or entirely of staple fibers assembled in a web, sheet or batt. The structure of the nonwoven fabric is based on the arrangement of, for example, staple fibers that are typically arranged more or less randomly. The tensile, stress-strain and tactile properties of the nonwoven fabric ordinarily stem from fiber to fiber friction created by entanglement and reinforcement of, for example, staple fibers, and/or from adhesive, chemical or physical bonding. Notwithstanding, the raw materials used to manufacture the nonwoven fabric may be yarns, scrims, netting, or filaments made by processes that include weaving or knitting.

Preferably, the nonwoven fabric is made by processes other than weaving or knitting. For example, the nonwoven fabric may be prepared from yarn, scrims, netting or filaments that have been made by processes that include weaving or knitting. The yarn, scrims, netting and/or filaments are crimped to enhance entanglement with each other. Such crimped yarn, scrims, netting and/or filaments may then be cut into staple that is long enough to entangle. The staple may be between about 0.1 and 2.5 inches long, preferably between about 0.5 and 1.75 inches, and most preferably between about 1.0 and 1.3 inches. The staple may be carded to create a nonwoven batt, which may be then needlepunched or calendared into a nonwoven fabric. Additionally, the staple may be kinked or piled.

Other methods known for the production of nonwoven fabrics may be utilized and include such processes as air laying, wet forming and stitch bonding.

Such procedures are generally discussed in the Encyclopedia of Polymer Science and Engineering, Vol. 10, pp. 204-253 (1987) and Introduction to Nonwovens by Albin Turbank (Tappi Press, Atlanta Ga. 1999), both incorporated herein in their entirety by reference.

The thickness of the nonwoven fabric may range from about 0.25 to 2 mm. The basis weight of the nonwoven fabric ranges from about 0.01 to 0.2 g/in²; preferably from about 0.03 to 0.1 g/in²; and most preferably from about 0.04 to 0.08 g/in².

One method of making the nonwoven fabric described herein is by the following process. Polymer fibers, having a denier per fiber of about 1 to 4, may be consolidated to about 80 to 120 denier multifilament yarn and then to about 800 to 1200 denier yarns, thermally crimped and then cut to a staple having a length between about 0.75 and 1.5 inch. The staple may be fed into a multiroller dry lay carding machine one or more times and carded into a uniform nonwoven batt, while humidity is controlled between about 20-60% at a room temperature of 15 to 24° C. For example, the uniform nonwoven batt may be made using a single cylinder rollertop card, having a main cylinder covered by alternate rollers and stripper rolls, where the batt is doffed from the surface of the cylinder by a doffer roller and deposited on a collector roll. The batt may be further processed via needlepunching or any other means such as calendaring.

The nonwoven fabric may be comprised of fibers comprising aliphatic polyester polymers, copolymers, or blends thereof. The aliphatic polyesters are typically synthesized in a ring opening polymerization of monomers including, but not limited to, lactic acid, lactide (including L-, D-, meso and D, L mixtures), glycolic acid, glycolide, ε-caprolactone, p-dioxanone (1,4-dioxan-2-one), and trimethylene carbonate (1,3-dioxan-2-one). Preferably, the nonwoven fabric comprises a copolymer of glycolide and lactide, in an amount ranging from about 70 to 95% by molar basis of glycolide and the remainder lactide.

In other embodiments, the dressing may comprise a gelatin sponge or a collagen sponge, since these substrates have voids that are capable of holding the proteins therein. Methods for preparing a gelatin or collagen sponge are described in U.S. Pat. No. 6,733,774.

The proteins described herein comprise blood protein/plasma protein. As used herein, the term “blood protein/plasma protein” refers to proteins found in blood plasma. The source of the proteins may be natural (i.e. human or animal), synthetic or recombinant. Blood protein/plasma protein serves as transport molecules for lipids, hormones, vitamins and metals. They also serve as enzymes, complement components, protease inhibitors, and kinin precursors. Blood protein/plasma proteins play an important role in the regulation of acellular activity and functioning and in the immune system. Separating serum proteins by electrophoresis is a valuable diagnostic tool as well as a way to monitor clinical progress. Blood protein/plasma protein includes, but is not limited to, albumin, ancrod, batroxobin, collagen, ecarin, elastin, epinephrine, Factor X/Xa, Factor VII/VIIa, Factor IX/IXa, Factor XI/XIa, Factor XII/XIIa, fibrin, ficolin, fibrinogen, fibronectin, gelatin, globin, haptoglobin, hemoglobin, heparinase, inhibin, insulin, interleukin, lamininthrombin, platelet surface glycoproteins, prothrombin, selectin, thrombin, transferin, von Willebrand Factor, vasopressin, vasopressin analogs, procoagulant venom, platelet activating agents and synthetic peptides having hemostatic activity.

Preferably, the protein is thrombin and/or fibrinogen, and may be animal derived, preferably human, or may be recombinant. The thrombin activity on the dressing may be in the range of about 20 to 500 IU/cm², preferably about 20 to 200 IU/cm², and more preferably about 30 to 200 IU/cm² and most preferably about 50 to 200 IU/cm². The fibrinogen activity on the dressing may be in the range of about 2 to 15 mg/cm², preferably about 3 to 10 mg/cm², and most preferably about 4 to 7 mg/cm².

In a preferred embodiment, the dressing retains solid thrombin and/or solid fibrinogen powder without separation and with minimal loss of the powder from its surface, and may be prepared as described herein. Thrombin and/or fibrinogen containing solutions are separately lyophilized. The lyophilized materials are then ground into powders using a superfine mill or a cooled blade mill. The powders are weighed and suspended together in a carrier fluid in which the proteins are not soluble. A preferred carrier fluid is a perfluorinated hydrocarbon, including but not limited to HFE-7000, HFE-7100, HFE-7300 and PF-5060 (commercially available from 3M of Minnesota). Any other carrier fluid in which the proteins do not dissolve may be used, such as alcohols, ethers or other organic fluids. The suspension is thoroughly mixed and applied to a dressing, such as a nonwoven fabric, via conventional means such as wet, dry or electrostatic spraying, dip coating, painting, or sprinkling, while maintaining a room temperature of about 15 to 24° C. and relative humidity of about 10 to 60%, preferably about 20 to 40%. The nonwoven fabric having the suspension thereon is then subjected to one or more applications of pressure ranging from about 2,500 to about 39,500 psi for about 2 to about 6 seconds, under conditions of temperature of less than about 30° C. and relative humidity ranging from about 20 to 60%. In the event that more than one applications of pressure is utilized, the pressure-treated nonwoven fabric may be allowed to cool to about 30° C. However, as contemplated by one skilled in the art, cooling to lower temperatures would allow for increased pressure to be applied upon successive applications of pressure. Such successive applications of pressure may include one or more steps of embossing a pattern onto the nonwoven fabric.

Preferably, the pressure applied to the dressing ranges from about 2,500 to about 39,500 psi, more preferably from about 4,000 to about 20,000 psi, for about 2 to about 6 seconds. The preferred conditions for temperature and humidity range from about 20 to 30° C. and a relative humidity of less than about 60%.

The dressing is then dried at ambient room temperature and packaged in a suitable moisture barrier container. The dressing having the thrombin and/or fibrinogen contains no more than about 25% moisture, preferably no more than about 15% moisture, and most preferably no more than about 5% moisture.

The amount of thrombin and/or fibrinogen powder applied to the nonwoven fabric is sufficient to cover its surface such that no area is visibly devoid of coverage. The powder may sit mostly on top of the nonwoven fabric or may penetrate into the nonwoven fabric.

As a surgical dressing, the dressing described herein may be used as an adjunct to primary wound closure devices, such as arterial closure devices, staples, and sutures, to seal potential leaks of gasses, liquids, or solids as well as to provide hemostasis. For example, the dressing may be utilized to seal air from tissue or fluids from organs and tissues, including but not limited to, bile, lymph, cerebrospinal fluids, gastrointestinal fluids, interstitial fluids and urine.

The dressing described herein has additional medical applications and may be used for a variety of clinical functions, including but not limited to tissue reinforcement and buttressing, i.e., for gastrointestinal or vascular anastomoses, approximation, i.e., to connect anastomoses that are difficult to perform (i.e. under tension), and tension releasing. The dressing may additionally promote and possibly enhance the natural tissue healing process in all the above events. This dressing can be used internally in many types of surgery, including, but not limited to, cardiovascular, peripheral-vascular, cardio-thoracic, gynecological, neuro- and general surgery. The dressing may also be used to attach medical devices (e.g. meshes, clips and films) to tissues, tissue to tissue, or medical device to medical device.

While the following examples demonstrate certain embodiments of the invention, they are not to be interpreted as limiting the scope of the invention, but rather as contributing to a complete description of the invention.

### COMPARATIVE EXAMPLE 1

Thrombin and fibrinogen containing fractions (obtained from Omrix Biopharmaceuticals (Israel) Ltd. Tel Hashomer, Israel) were prepared by removing the liquid component via lyophilization to form individual dry powder bricks of thrombin and fibrinogen. The bricks were broken, and then feed into a jet mill (Super Fine Vortex Mill, Super Fine LTD, Yokneam, Israel) to form particulate powder. The particulate powder was then suspended in a hydrofluoroether solvent (HFE-7000 obtained from 3M, Minnesota) with continuous agitation using a peristaltic pump in recirculation mode (Marlow & Watson Bredel, USA). The resulting suspension was applied by spraying onto one side of a multilayered substrate comprising a first absorbable non-woven fabric of a glycolide/lactide copolymer reinforced by a knitted oxidized regenerated cellulose fabric, using a spray nozzle that is moved in a steady motion over the nonwoven fabric to deposit the powders in a uniform fashion, followed by drying the solvent over time. Care was taken to insure that the coated substrate was not exposed to moist conditions. The coated substrate was placed into a moisture barrier pouch (SCC Dri-Shield 3M). The pouch was then placed in a carver press (Fred S. Carver Press Company, Wabash, Ind.) and subjected to 10,000 pounds of force on the material, to exert a pressure of about 2,000 psi, for 5 seconds. The pouch was opened under low humidity conditions (less than 40%) and visually inspected. The coated substrate was initially flat, however the powder was not transformed into a uniform film.

### INVENTIVE EXAMPLE 1

The coated substrate from Comparative Example 1 was then placed back into the pouch and sealed. A pressure of 2,500 psi was exerted on the coated substrate for 5 seconds. The coated substrate was visually observed to be flat with the thrombin and fibrinogen in a homogenous film.

### INVENTIVE EXAMPLE 2

A 2×3 inch sample of a multilayered substrate comprising an absorbable nonwoven fabric of a glycolide/lactide copolymer reinforced by a knitted oxidized regenerated cellulose fabric, having a suspension of thrombin and/or fibrinogen sprayed thereon as described in Comparative Example 1, was placed under 3,300 psi of pressure for 3 seconds, in a low moisture environment, i.e., less than 40% humidity. This sample was evaluated by SEM, which showed streams of melted or dissolved material present among the powdered coating. The coated surface had a harder and more defined two dimensional surface, rather then a fragile “powdery” surface. The distribution of the thrombin/fibrinogen coating was largely confined to the surface. Very little evidence of penetration into the nonwoven fabric was observed.

### INVENTIVE EXAMPLE 3

A 2×3 inch sample of a multilayered substrate comprising an absorbable nonwoven fabric of a glycolide/lactide copolymer reinforced by a knitted oxidized regenerated cellulose fabric, which was coated by spraying a suspension of thrombin and fibrinogen suspended in HFE, was placed in a foil pouch and then under 3,300 psi of pressure for 3 seconds. Upon visual inspection, the material looked uniform. The sample was then cut into a circular disk using a die that punched out a circle of approximately 20 mm in diameter. Very little shedding of the thrombin and fibrinogen was observed during the punching process. By contrast, powder was dislodged from a sample that had not undergone a pressure application to fixate the powder as observed in Comparative Example 1.

### INVENTIVE EXAMPLE 4

A 2×3 inch sample of multilayered substrate comprising an absorbable nonwoven fabric of a glycolide/lactide copolymer reinforced by a knitted oxidized regenerated cellulose fabric was coated by immersion of the substrate into a suspension of thrombin, fibrinogen and HFE. The coated substrate was subjected to 3,000 psi of pressure for 5 seconds to produce a homogenous non-shedding dressing, under visual inspection. A portion of the sample was punched into a disk, which resulted in almost no flaking being observed.

### INVENTIVE EXAMPLE 5

A 2×3 inch sample of multilayered substrate comprising an absorbable nonwoven fabric of a glycolide/lactide copolymer reinforced by a knitted oxidized regenerated cellulose fabric was coated by immersion of the substrate into a suspension of thrombin, fibrinogen and HFE. Three samples of this coated substrate were subjected to 3,000 psi of pressure for 5 seconds. The three pressure-treated samples were embossed by placing a piece of suture 4.0 monofilament on the pressure-treated sample and subjected to 1,500 psi of pressure.

### INVENTIVE EXAMPLE 6

Three samples, Samples 6A-C, prepared from a coated substrate, as described in Inventive Example 5, were treated with a pressure of 4,500 psi for 5 seconds to fixate the thrombin and fibrinogen onto the substrate. Upon visual inspection, the pressure treatment caused the thrombin and fibrinogen to have a more uniform and one-dimensional appearance compared to coated substrates samples that were not subjected to the pressure treatment, Samples 6D-E. Such pressure treatment resulted in a coated substrate that is malleable without evidence of significant flaking or cracking.

Each sample was placed in a pre-weighted glass scintillation vial and was dropped from 4 feet to a rubber mat on the floor. The vials were allowed to bounce and finally come to rest with the drop being repeated to help normalize the stress each sample was exposed to. After the drops, the sample was removed from the scintillation vial and weighed. The increase in vial weight was the result of thrombin and fibrinogen being shed from the sample. The change in weight of the sample was reported as a percentage of the total sample weight before the drop.

TABLE 1

| | Weight loss Fleece | Initial weight of | |
|---|---|---|---|
| Sample# | (Grams) | Fleece (Grams) | % weight change |
| 6A. | 0.153 | 0.2312 | 6.6% |
| 6B. | 0.0081 | 0.2356 | 3.4% |
| 6C. | 0.0089 | 0.2446 | 3.6% |
| 6D. | 0.0307 | 0.2527 | 12.1% |
| 6E. | 0.0359 | 0.215 | 16.7 |

### It is demonstrated that pressure treated Samples 6A-C experienced a reduced level of loss of thrombin and fibrinogen powders compared to non-pressure treated Samples 6D-E.

### INVENTIVE EXAMPLE 7

Three samples 2×2 inch of multilayered substrate comprising an absorbable non-woven fabric of a glycolide/lactide copolymer reinforced by a knitted oxidized regenerated cellulose fabric, Samples 7A-C, were prepared as described in Inventive Example 5, then treated with pressure (7A: 4,000 psi/5 second, 7B: 4,500 psi/5 second, 7C: 4,500 psi/5 second) to fixate the thrombin and fibrinogen onto the substrate. Samples 7A was subsequently treated with a pressure of 2,500 psi for 5 second to emboss with a graphic design as described in Example 5. The pressure treatment caused the thrombin and fibrinogen to have a more uniform and one-dimensional appearance. No flaking or shedding of the biologic powder was visually observed. These materials exhibited moderate to high adhesive characteristics and provided the expected hemostatic function in an aortic punch hemostasis model for brisk bleeding.

### COMPARATIVE EXAMPLE 8

Three samples 2×2 inch of multilayered substrate comprising an absorbable non-woven fabric of a glycolide/lactide copolymer reinforced by a knitted oxidized regenerated cellulose fabric, Sample 8A-C, were prepared as described in Inventive Example 5, then treated with pressure (8A: 40,000 psi/5 second, 8B: 100,000 psi/5 second, 8C: 200,000 psi/5 second) to fixate the thrombin and fibrinogen onto the substrate. The extreme high pressure treatment caused the thrombin and fibrinogen to appear glassy and brittle and the coated substrate to have poor handability.

While the examples demonstrate certain embodiments of the invention, they are not to be interpreted as limiting the scope of the invention, but rather as contributing to a complete description of the invention.

We claim:

1. A method for making a dressing having at least one protein, comprising the steps of:
(a) applying said at least one protein to said dressing; and
(b) subjecting said dressing having at least one protein thereon to pressure ranging from about 2,500 to about 39,500 psi for about 2 to about 6 seconds.

2. The method according to claim 1, where the dressing comprises a knitted, woven or nonwoven fabric, a gelatin sponge or a collagen sponge.

3. The method according to claim 2, wherein the fabric comprises fibers comprised of aliphatic polyester polymers or copolymers of one or more monomers selected from the group consisting of lactic acid, lactide (including L-, D-, meso and D, L mixtures), glycolic acid, glycolide, ε-caprolactone, p-dioxanone, and trimethylene carbonate.

4. The method according to claim 3, where the fabric comprises glycolide/lactide copolymer.

5. The method according to claim 1, where the protein is selected from the group consisting of thrombin, fibrinogen, fibrin, albumin, transferin, and plasmin.

6. The method according to claim 5, where the proteins are thrombin and fibrinogen.

7. The method according to claim 6, wherein the thrombin activity on the dressing ranges from about 20 to 500 IU/cm², and the fibrinogen activity on the dressing ranges from about 2 to 15 mg/cm².

8. The method according to claim 1, wherein step (b) is conducted at a temperature of less than 150° C. and relative humidity ranging from about 10 to 60%.

9. The method according to claim 8, wherein step (b) is conducted at a temperature ranging from 20 to 25° C. and a relative humidity ranging from about 20% to 40%.

10. The method according to claim 9, wherein said at least one protein is applied to said dressing via wet, dry or electrostatic spraying, dip coating, painting, or sprinkling a suspension of said at least one protein onto said dressing.

11. A method according to claim 2, wherein the dressing comprises a gelatin sponge or a collagen sponge, and the protein is selected from the group consisting of thrombin, fibrinogen and fibrin.

12. A method for making a dressing having at least one absorbable nonwoven fabric comprising glycolide/lactide copolymer and at least one protein selected from the group consisting of thrombin, fibrinogen and fibrin, comprising the steps of:
(a) applying said at least one protein to said absorbable nonwoven fabric; and
(b) subjecting said absorbable nonwoven fabric having at least one protein thereon to pressure ranging from about 2,500 to about 39,500 psi for about 2 to about 6 seconds, at a temperature ranging from about 20 to 25° C. and a relative humidity ranging from about 20% to 40%.
